(19) United States
(12) Patent Application Publication
(10) Pub. No.: US 2014/0322149 A1
(43) Pub. Date: Oct. 30, 2014

(54) COMPOUND USEFUL FOR TREATING CELLULITE

(71) Applicant: SIGMA-TAU INDUSTRIE FARMACEUTICHE RIUNITE S.P.A., Rome (IT)

(72) Inventor: Aleardo KOVERECH, Rome (IT)

(21) Appl. No.: 14/328,803

(22) Filed: Jul. 11, 2014

Related U.S. Application Data
(62) Division of application No. 13/121,922, filed on May 26, 2011.

(30) Foreign Application Priority Data
Nov. 11, 2008 (EP) 08168795.6

(51) Int. Cl. A61K 31/225; A61K 45/06; A61K 8/37; A61K 8/35; A61Q 19/02; A61Q 19/08; A61Q 17/04; A61K 8/44; A61Q 19/06

(52) U.S. Cl. CPC A61K 31/225; A61K 8/44; A61K 45/06; A61K 8/37; A61Q 19/06; A61Q 19/02; A61Q 19/08; A61Q 17/04; A61K 8/35; A61K 8/445
USPC 424/60; 514/547; 424/59

(57) ABSTRACT

The use of propionyl L-carnitine for treating disturbances of the skin such as cellulite is described.

### CROSS REFERENCE TO RELATED APPLICATIONS

This application is a divisional of U.S. application Ser. No. 13/121,922 filed on May 26, 2011, which is a 371 U.S. national phase of International Application No. PCT/EP2009/064681 filed on Nov. 5, 2009, which claims priority to European Application No. 08168795.6 filed on Nov. 11, 2008, the contents of which are incorporated herein by reference.

The present invention relates to a composition, intradermally administrable, useful for treating disturbances of the skin.

### FIELD OF THE INVENTION

In particular, the present invention relates to a composition comprising as active ingredient propionyl L-carnitine or a salt thereof, for pharmaceutical or cosmetic use, useful for preventing or treating cellulite.

The distribution of adipose tissue throughout the body is not uniform. In certain portions of the body it is present in great abundance such as in the subcutaneous tissue. A distinction must be made between fat and adipose tissue; the latter being a distinct tissue, the former an oily substance. Adipose tissue consists of small vesicles referred to hereinafter as “fat cells” lodged within the matrix of areolar connective tissue. Fat cells vary greatly in size; having an approximate diameter of about 0.05 mm. They are formed of a delicate protoplasmic membrane filled with the oily substance which is liquid during life but solidifies after death. These fat cells are contained in discrete clusters in the areolae of fine connective tissue.

Areolar tissue is a form of connective tissue wherein the investing connective tissue matrix is separated into areolae or spaces which open into one another and are easily permeated by fluids. Areolar tissue binds different parts of the body together. The elasticity of areolar tissue and the permeability of its areolae allows the various parts of the body to move relative to one another. Most particularly, areolar connective tissue is found beneath the skin in a continuous layer all over the body, connecting the skin (dermis) to subjacent tissues. In many parts the areolae are occupied by fat cells; the matrix and fat cells constituting adipose tissue which is referred to alternatively herein as “depot fat”.

Cellulite is typically characterized by dermal deterioration due to a breakdown in blood vessel integrity and a loss of capillary networks in the dermal and subdermal levels of the skin. The vascular deterioration tends to decrease the dermal metabolism. This decreased metabolism hinders protein synthesis and repair processes, which results in dermal thinning. The condition is further characterized by fat cells becoming engorged with lipids, swelling, and clumping together, as well as excess fluid retention in the dermal and subdermal regions of the skin. Thus, individuals afflicted with cellulite tend to have a thicker subcutaneous fatty layer of skin. In the advanced stages of cellulite, reticular protein deposits called septa begin to form around the fatty deposits in the skin and occlude the fat cells. As the condition further progresses, hard nodules of fat cells and clumps of fat surrounded by septa form in the dermal region. This leads to the surface of the skin displaying considerable heterogeneity and being characterized as having a “cottage cheese” appearance. This appearance is most pronounced in overweight individuals. Individuals with cellulite also tend to have a thinner epidermis and dermis in the affected region, decreased firmness of the skin, and decreased rate of cell renewal.

There is no quick fix solution for cellulite reduction, and the obvious and most inexpensive way to treat cellulite is to watch what we eat and drink, and burn those calories by exercising on a regular basis.

Thousands of OTC potions, creams and pills to combat cellulite have flooded the market but the fact remains that cellulite is still stubborn and refuses to budge easily.

The appearance of cellulite currently tends to be treated by administering xanthines, which include caffeine, theophylline, and aminophylline. Xanthines acts as a diuretic that removes water from the fat cells and thus reduces the size of the fat cells. The effect of xanthines, however, is temporary and the fat cells become rehydrated as soon as the individual replenishes the lost water.

A variety of vitamins and minerals have individually been administered to treat certain skin and other problems that occur when the patient has a deficiency of that vitamin or mineral. Vitamin A, for example, assists in the treatment of acne and to facilitate wound healing; vitamin C (ascorbic acid) assists in the prevention of skin bruising and wound healing; vitamin E is an antioxidant; and copper assists in the treatment of elastic tissue defects. [Neldner, K. H., Amer. Acad. Derm. Annl. Mtg., Wash. D.C., Dec. 6, 1993]. Topical use of vitamin C is also believed to ward off sun damage, reduce breakdown of connective tissues, and possibly promote collagen synthesis. [Dial, W., Medical World News, p. 12, March 1991]. Vitamin E is used intradermally as an anti-inflammatory agent, for enhancement of skin moisturization, for UV-ray protection of cells, and for retardation of premature skin aging.

In spite of the large number of products useful for treating skin disturbances, in the medical and cosmetic field it is still a perceived need to have new active ingredients, useful for preparing cosmetic compositions for the preventing or treating skin disturbances.

### DETAILED DESCRIPTION OF THE INVENTION

It has now been found that propionyl L-carnitine is a useful agent for the prevention or treatment of disturbances of the skin.

It is therefore an object of the present invention a composition, topically or intradermally administrable, for pharmaceutical or cosmetic use, comprising as active ingredient propionyl L-carnitine or a pharmaceutically salt thereof.

What is meant by pharmaceutically acceptable salt of propionyl L-carnitine is any salt of the latter with an acid that does not give rise to toxic or side effects.

These acids are well known to pharmacologists and to experts in pharmacy. Non-limiting examples of such salts are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

What is meant by pharmaceutically acceptable salt of propionyl L-carnitine is also a salt approved by the FDA and listed in the publication Int. J. of Pharm. 33 (1986), 201-217, which is incorporated herein by way of a reference.

It is a further object of the present invention propionyl L-carnitine for use as anticellulitic agent.

It is a further object of the present invention the use of propionyl L-carnitine or a salt thereof, for preparing a pharmaceutical or cosmetic composition, topically or intradermally administrable, useful for: supporting the fibrous matrix layer of tissue beneath the skin; preventing subdermal tissue from entering or protruding into to the dermis; treating patients having cellulite; or for the purpose of causing the contraction of laxed or wrinkled tissues below the surface of the epidermis.

It is a further object of the present invention the use of propionyl L-carnitine or a salt thereof, for preparing a pharmaceutical or cosmetic composition, topically or intradermally administrable, for the prevention or treatment of cellulite.

It is a further object of the present invention the use of propionyl L-carnitine or a salt thereof, for preparing a pharmaceutical or cosmetic composition useful for the prevention or treatment of cellulite, in which said propionyl L-carnitine is administered intradermally once per week for at least 5 weeks.

It is a further object of the present invention the use of propionyl L-carnitine or a salt thereof, for preparing a pharmaceutical or cosmetic composition, topically or intradermally administrable, for the prevention or treatment of heaviness in the legs.

The pharmaceutical or cosmetic composition of the invention may further comprise one or more excipients or diluent and/or one or more of the following cosmetically acceptable ingredients:

a) a suitable surfactant selected from: sodium dodecyl sulphate; amino acid based cationic surfactant made from, for example, L-arginine, DL-pyrrolidone carboxylic acid, coconut fatty acids; or amino acid-based nonionic surfactants; and

b) at least one active ingredient useful for the prevention or treatment of disturbances of the skin selected from:
agents supporting the microcirculation which include, but are not limited to, extracts of Gingko biloba, ruscus, melilot, red vine, viburnum;
agents for the activation of the lipolysis which include, but are not limited to, extracts of Ground ivy (Glechoma), root of Angelica, extract of Paulinia, Subdued or of the xanthic bases such as cafeine, theobromine and theophylline;
anti-inflammatory compounds which include, but are not limited to, rosmarinic acid, glycyrrizinate derivatives, alpha bisabolol, azulene and derivatives thereof, asiaticoside, sericoside, ruscogenin, escin, escolin, quercetin, rutin, betulinic acid and derivatives thereof, catechin and derivatives thereof;
skin whitening compounds which include, but are not limited to, ferulic acid, hydroquinone, arbutine, and kojic acid;
antioxidants and anti-wrinkling compounds which include, but are not limited to, retinol and derivatives, tocopherol and derivatives, salicylates and their derivatives;
agents which improve skin penetration and efficacy of common anticellulite agents which include, but are not limited to a monocarboxylic acids comprising lactic acid, glycolic acid, mandelic acid and mixtures thereof;
essential fatty acids (EFAs) exerting an important role in skin defence against oxidative stress, by entering in the lipid biosynthesis of epidermis and providing lipids for the barrier formation of the epidermis; preferred essential fatty acids are selected from the group consisting of linoleic acid, gamma-linolenic acid, homo-gamma-linolenic acid, columbinic acid, eicosa-(n-6,9,13)-trienoic acid, arachidonic acid, gamma-linolenic acid, timnodonic acid, hexaenoic acid and mixtures thereof; or
sunscreens, for example, derivatives of Para Amino Benzoic Acid (PABA), cinnamate and benzophenone derivatives such as octyl methoxy-cinnamate and 2-hydroxy-4-methoxy-benzophenone;

c) optionally at least one excipient or diluent selected from:
thickener agents in any suitable proportion well known to the skilled in the art; exemplary thickener agent are gums such as xanthan, carrageenan, gelatin, karaya, pectin and locust beans gum; said water-based cosmetic composition can be protected;
preservatives against the growth of microorganisms; suitable preservatives include alkyl esters of p-hydroxybenzoic acid, hydantoin derivatives, propionate salts, methyl paraben, propyl paraben, imidazolidinyl urea, sodium dehydroxyacetate benzyl alcohol, and a variety of quaternary ammonium compounds. Preservatives, if any, are added any suitable proportion well known to the skilled in the art;
silicone polymers in any suitable proportion well known to the skilled in the art;
emollients acting both as carrier, to facilitate the dispersion of the active ingredient and skin softners; emollients may be incorporated in the cosmetic composition of the invention in any suitable proportion well known to the skilled in the art; suitable emollients may be classified under such general chemical categories as esters, fatty acids and alcohols, polyols and hydrocarbons; an example of fatty di-esters include: dibutyl adipate, diethyl sebacate, diisopropyl dimerate, propylene glycol myristyl ether acetate, diisopropyl adipate, and dioctyl succinate; an example of branched chain fatty esters include 2-ethyl-hexyl myristate, isopropyl stearate and isostearyl palmitate; an example of tribasic acid esters include triisopropyl trilinoleate, trilauryl citrate, tributirrine, and saturated or unsaturated vegetable oils; an example of straight chain fatty esters include lauryl palmitate, myristyl lactate, oleyl eurcate, stearyl oleate coco-caprylate/caprate, and cetyl octanoate; an example of fatty alcohols and acids are C₁₀-C₂₀ compounds such as cetyl, myristyl, palmitic and stearyl alcohols and acids; an example of polyols are linear and branched chain alkyl polyhydroxyl compounds, such as propylene and butylene glycol, sorbitol glycerin, as well as polymeric polyols such as polypropylene glycol and polyethylene glycol; an example of hydrocarbons are linear C₁₂-C₃₀ hydrocarbon chains such as mineral oil, petroleum jelly, squalene and isoparaffins;
water;
colouring agents,
opacifiers;
perfumes.

According to the present invention propionyl L-carnitine in the form of cosmetic or pharmaceutical composition is administered intradermally or topically in the form of liquid, cream or lotion, comprising from 0.5 to 45% by weight, preferably from 5 to 35% by weight, more preferably from 10 to 25% by weight of active ingredient, optionally in admixture with suitable customary auxiliary agents. The preferred dose to be administered topically in the form of cream is 20%.

The preferred dose to be administered intradermally using, for example a “Lebel niddle”, is 50 mg of propionyl L-carnitine in 2.5 mL of steril demineralised water.

The topical skin treatment composition of the invention can be formulated in all the forms topics used in beauty care: lotion, fluid cream, cream or gel. The composition can be packaged in a suitable container according to its viscosity and to the intended use by the user. For example, a lotion or fluid cream can be packaged in a bottle, in a roll-ball applicator, in a capsule, patch, in a propellant-driven aerosol device or a container fitted with a pump suitable for finger operation.

When the composition is a cream, it can simply be stored in a non-deformable bottle or in a squeeze container, such as a tube or a lidded jar.

For each particular form, one has recourse to suitable excipients.

These excipients must have all usually required qualities. As examples, one can quote: the propylene glycol, the glycerin, cetyl alcohol, the polyols, the phospholipides put in liposomes or not, oils vegetated, animal, mineral, preservatives, the dampeners, the thickeners, stabilizing and emulsifying usually used.

The expression “cosmetically acceptable ingredients” according to the present invention are products which are suitable for their use in cosmetic treatments, for example those included in the INCI list drawn by the European Cosmetic Toiletry and Perfumery Association (COLIPA) and issued in 96/335/EC “Annex to Commission Decision of 8 May 1996”.

### EXAMPLE 1

### Topical Anticellulite Activity of Propionyl L-Carnitine

The effectiveness of intradermally administration of the product according to the invention was tested for its ability to reduce the appearance of cellulite in the thigh. A total of 54 female subjects ranging in age from 23 to 58 years of age were selected to evaluate the composition of the invention.

The subjects were selected based on their cellulite intensity in the thigh area having a bi-lateral symmetry. Subjects with grades 1 and 2 cellulite were chosen, as a 5-point grading scale was used to rate the cellulite severity of each subject. The scale ranged from 0 to 4, being 0=No cellulite; 1=Small bumps or depressions; 2=Striations and bumps; 3=Pronounced lumpiness of the skin and striations; 4=All of the above plus hard sub-surface nodules.

All subjects had the absence of any visible skin disease(s) which might be confused with a skin reaction from the test material and were in general good health with no known allergies, especially to cosmetic or toiletry products; had no evidence of acute or chronic disease; were not pregnant or lactating; were not on any diet or weight reduction program; and were not on any regular exercise program (immediately prior to or during the course of the study).

At baseline each subject received a visual examination conducted by a qualified technician and scored for the degree of cellulite. In the same day subjects were treated on the right and left thigh intradermally by multiple injection, (from 5 to 10 injections per thigh using a “Lebel needle” 4 mm×0.4 mm; 27 G). The treatment was repeated once per week for five consecutive weeks (week 0; 1; 2; 3; 4; and 5).

The degree of cellulite was evaluated according to the following scale: 0=No visible cellulite; 1=Very little visible cellulite, no dimpling; 2=Visible cellulite, evidence of shallow dimpling; 3=Easily visible cellulite, moderate to pronounced dimpling; 4=Extremely visible cellulite, heavy and deep dimpling.

The patients were divided in 3 groups and treated intradermally by multiple injection with a composition comprising:

Group A
Propionyl L-Carnitine: 50.0 mg;
Mannitol: 100.0 mg;
Sodium phosphate bibasic bi-hydrate: 24.0 mg;
Trometamine: 8.0 mg;
H₂O: 2.5 mL (final volume);

Group B
Acetyl L-Carnitine: 50.0 mg;
Mannitol: 100.0 mg;
Sodium phosphate bibasic bi-hydrate: 24.0 mg;
Trometamine: 8.0 mg;
H₂O: 2.5 mL (final volume); or

Group C
L-Carnitine: 50.0 mg;
Mannitol: 100.0 mg;
Sodium phosphate bibasic bi-hydrate: 24.0 mg;
Trometamine: 8.0 mg;
H₂O: 2.5 mL (final volume).

Subjects were instructed to discontinue the use of their normal anti-cellulite products to avoid introducing any new products for treating cellulite during the study, and to not be on any diet or weight reduction program or on any regular exercise program immediately prior to or during the course of the study. Each subject was also instructed to keep a diary to document compliance.

After 7 days from the last treatment (week 6) the subjects returned to the hospital for a final visual evaluation.

The cellulite evaluation was made according the method described in Cosmetics & Toiletries, 61-70, June 1995, by comparison of the values before and after the treatment.

During the treatment period none of the subject reported adverse events such as itching and/or redness.

Results obtained are reported in Table 1.

TABLE 1
Change of the cellulite condition after 6 weeks application

| | A | B | |
|---|---|---|---|
| End of | Propionyl | Acetyl | C |
| treatment | L-carnitine | L-carnitine | L-carnitine |
| Reduction of | −11% | −2% | −3% |
| the thigh | | | |
| diameter | | | |
| P< vs base line | 0.01 | ns | Ns |
| Reduction of | −28% | −10% | −12% |
| the fatty layer | | | |
| thickness | | | |
| P< vs base line | 0.001 | 0.05 | 0.05 |
| Skin firmness | +20% | +6% | +9% |
| P< vs base line | 0.01 | NS | NS |
| Skin hydration | +27% | +8% | +10% |
| P< vs base line | 0.001 | 0.05 | 0.05 |
| Surface | +42% | +15% | +18% |
| smoothness | | | |
| P< vs base line | 0.001 | 0.05 | 0.05 |
| Subjective | +57% | +15% | +19% |
| improvement | | | |
| Clinical | +34% | +8% | +12% |
| grading | | | |
| Irritation | 0 | 0 | 0 |
| reactions | | | |

The results reported in Table 1 show that (a) propionyl L-carnitine effectively ameliorate the cellulite condition; (b) the activity of L-carnitine or acetyl L-carnitine was inferior compared with that of propionyl L-carnitine; (c) propionyl L-carnitine improved skin infrastructure by beneficially affecting the dermis of the skin.

The activity of the compound of the invention was also significantly higher respect to the activity of L-carnitine or acetyl L-carnitine, and these results were unexpected.

### EXAMPLE 2

### Heaviness in the Legs

15 Patients with symptoms of heaviness in the legs (belonging to group A of Example 1) were scored both, before the beginning of the treatment and after 5 weeks of treatment, using a visual analog scale (VAS).

The visual analog scale (VAS) consists of a 10-cm line anchored by two extremes. Patients were asked to make a mark on the line that represented their level of discomfort with the 0 value indicating the “absence of feeling of heaviness in the legs” and 100 to indicate “maximum feeling of heaviness in the legs”. The measurement was taken before the application at T0 and after 5 weeks of treatment.

The patients with symptoms of heaviness in the legs treated with the composition of the invention reported a significant reduction in the intensity of the symptoms respect to the basal values.

Propionyl L-carnitine is a known compound and its preparation process is described in U.S. Pat. No. 4,254,053.

The pharmaceutical or cosmetic compositions according to the present invention are composed of active ingredients which are familiar to operators in the medical or cosmetic field, already in use and their toxicological profiles already known.

Their procurement therefore is very easy, inasmuch as these are products which have been on the market now for a long time and are of a grade suitable for human administration.

An example of composition of the invention for intradermally injection is reported in the following.

### Composition 1

| Lyophylised powder | Diluent |
|---|---|
| Propionyl L-Carnitine HCl: mg 50.0 | Sodium phosphate |
| Mannitol: mg 100.0 | bibasic bi-hydrate: mg |
| | 24.0 |
| | Trometamine: mg 8.00 |
| | H₂O: 2.5 ml (final volume). |

1. A method for preventing or treating cellulite, comprising topically or intradermally administering an anticellulitic composition comprising an effective amount of propionyl L-carnitine or a salt thereof to subject in needed thereof.

2. The method of claim 1, wherein the salt of propionyl L-carnitine is selected from the group consisting of: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, 2 magnesium tartrate, 2-amino-ethanesulphonate, magnesium 2-amino-ethanesulphonate, methanesulphonate, choline tartrate, trichloroacetate and trifluoroacetate.

3. The method of claim 1, in which propionyl L-carnitine is administered intradermally once per week for at least 5 weeks.

4. The method of claim 1, further comprising administering at least one active ingredient selected from the group consisting of:
agents supporting the microcirculation;
agents for the activation of the lipolysis;
anti-inflammatory compounds;
skin whitening compounds;
antioxidants and anti-wrinkling compounds;
agents which improve skin penetration and efficacy of common anticellulite agents;
essential fatty acids (EFAs); and
sunscreens.

5. The method according to claim 1, wherein the agents supporting the microcirculation are selected from the group consisting of: extracts of Gingko biloba, ruscus, melilot, red vine and viburnum.

6. The method according to claim 1, wherein the agents for the activation of the lipolysis are selected from the group consisting of: extracts of Ground ivy, root of Angelica, Paulinia; or of xanthic bases such as cafeine, theobromine and theophylline.

7. The method according to claim 1, wherein the anti-inflammatory compounds are selected from the group consisting of: rosmarinic acid, glycyrrizinate derivatives, alpha bisabolol, azulene and derivatives thereof, asiaticoside, sericoside, ruscogenin, escin, escolin, quercetin, rutin, betulinic acid and derivatives thereof, catechin and derivatives thereof.

8. The method according to claim 1, wherein the skin whitening compounds are selected from the group consisting of: ferulic acid, hydroquinone, arbutine, and kojic acid.

9. The method according to claim 1, wherein the antioxidants and anti-wrinkling compounds are selected from the group consisting of: retinol and derivatives thereof, tocopherol and derivatives thereof, salicylates and their derivatives thereof.

10. The method according to claim 1, wherein the agents which improve skin penetration and efficacy of common anticellulite agents are selected from the group consisting of: a monocarboxylic acids comprising lactic acid, glycolic acid, mandelic acid and mixtures thereof.

11. The method according to claim 1, wherein the essential fatty acids (EFAs) are selected from the group consisting of: linoleic acid, gamma-linolenic acid, homo-gamma-linolenic acid, columbinic acid, eicosa-(n-6,9,13)-trienoic acid, arachidonic acid, timnodonic acid, hexaenoic acid and mixtures thereof.

12. The method according to claim 1, wherein the sunscreens are selected from the group consisting of: PABA and derivatives thereof, cinnamate and benzophenone derivatives such as octyl methoxy-cinnamate and 2-hydroxy-4-methoxy-benzophenone.

13. The method according to claim 1, wherein the propionyl L-carnitine is present in an amount from 0.5 to 45% by weight.

14. Method according to claim 1, wherein the propionyl L-carnitine is present in an amount from 5 to 35% by weight.

15. The method according to claim 1, wherein the propionyl L-carnitine is present in an amount from 10 to 25% by weight.

16. The method according to claim 1, wherein the propionyl L-carnitine is present in an amount of 20% by weight.

17. A method for preventing or treating cellulite, comprising topically or intradermally administering an anticellulitic composition consisting of 50.0 mg of propionyl L-carnitine or a salt thereof, 100.0 mg mannitol, 24.0 mg sodium phosphate bibasic bi-hydrate, 8.0 mg trometamine and water up to a final volume of 2.5 ml.

18. Composition, topically or intradermally administrable, comprising as active ingredient propionyl L-carnitine or a salt thereof and optionally one or more excipient and/or diluent.

19. Composition according to claim 18, in which the salt of propionyl L-carnitine is selected from the group consisting of: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.
